(19) United States
(12) Patent Application Publication
(10) Pub. No.: US 2006/0040860 A1
(43) Pub. Date: Feb. 23, 2006

(54) Intra-tracheal application of vascular endothelial growth factor (VEGF) for the prevention of lung damages caused by hperoxia

(76) Inventors: Mario Ruediger, Innsbruck (AT); Edda Tschirch, Innsbruck (AT); Bernd Ruestow, Berlin (DE)

Correspondence Address: ROTHWELL, FIGG, ERNST & MANBECK, P.C., 1425 K STREET, N.W., SUITE 800, WASHINGTON, DC, 20005, US

(21) Appl. No.: 11/154,584

(22) Filed: Jun. 17, 2005

Related U.S. Application Data
(60) Provisional application No. 60/580,110, filed on Jun. 17, 2004.

(51) Int. Cl. A61K 38/18; A61L 9/04

(52) U.S. Cl. 514/12; 424/45

(57) ABSTRACT

The invention relates to the field of pulmonary diseases that are related to oxygen provision that is higher than physiological (hyperoxia) and to the use of vascular endothelial growth factor (VEGF) for treatment and prevention. Hyperoxic exposure causes a direct cellular damage in the lung and disturbs lung development. The effect becomes clinically prominent by the development of acute or chronic lung disease. There is a need for a pharmaceutical to prevent oxygen induced lung injuries. VEGF is not only a cellular survival factor but also important for lung development. The oxygen concentration regulates pulmonary VEGF expression, with a suppression during hyperoxia. Low pulmonary VEGF concentrations are responsible for oxygen induced effects. According to the present invention VEGF is used as a medicament for the treatment and/or prevention of pulmonary diseases or conditions that are related to high concentrations of inspired oxygen.

The present invention relates to the field of pulmonary diseases or conditions that are related to a deregulation of the oxygen provision and more specifically to the use of vascular endothelial growth factor (VEGF) for the treatment and prevention of these pulmonary diseases or conditions. The pulmonary diseases or conditions treated or prevented, respectively, are caused by high concentrations of inspired oxygen (hyperoxia).

Oxygen is the basic requirement for human life. However, in case that oxygen is used at supra-physiological concentrations it can also damage the human organism. For example, oxidative stress can cause acute and chronic lung injury. The prolonged exposure to a high concentration of oxygen, such as during mechanical ventilation, represents a necessary life-saving therapy for critically ill patients, but does also induce oxidative stress to the lung and causes pulmonary oxygen toxicity. Due to hyperoxia, i.e. the abnormal increase in the amount of inspired oxygen, the lungs can be acutely injured, subsequently leading to chronic damages of the lungs. To develop therapeutic strategies that can prevent these injuries, the underlying pathways of hyperoxia-induced pulmonary damage have to be understood.

In preterm infants, a “relative hyperoxic” exposure occurs post-nataly. Normal development in utero takes place at low oxygen concentration. Exposure of the preterm infant to room air (oxygen concentration of 21%) causes a higher oxygen concentration than during fetal life. In this term the “relative hyperoxic” condition represents a higher oxygen concentration than under normal physiological situations. Since many developmental processes depend on the oxygen concentration, an alteration of normal development can be expected.

Especially during long lasting hyperoxic exposure a direct cellular damage occurs in the lung accompanied by a destruction of the alveolar epithelium, perturbed gas exchange and development of a lung edema (1). Furthermore, hyperoxia induces apoptotic changes (leading to cellular death) in the lung by either activating or inhibiting different cytokines (2). If other damaging factors occur simultaneously, such as mechanical ventilation with baro- or volutrauma or a vitamin E deficiency, an imbalance of the oxidative/anti-oxidative system will lead to an increased apoptosis and a subsequent damage of pulmonary cells (3).

The hyperoxia-induced damage of the lung becomes clinically prominent by the development of an acute or chronic lung disease. High concentrations of inspired oxygen facilitate the progress of the acute respiratory distress syndrome (ARDS) in adults. In the premature lung of the preterm infant a long-term mechanical ventilation and highly concentrated oxygen supplementation lead to the development of a chronic lung disease, the broncho-pulmonary dysplasia (BPD) (4).

1.8% of all neonates suffer from a respiratory insufficiency that requires mechanical ventilation and oxygen supplementation. All together about 80,000 neonates per year are affected in USA. Whereas respiratory insufficiency is only a minor problem in term infants, almost all very preterm infants suffer from respiratory distress. Even though it was possible to decrease the lethality of the extremely premature infants, the incidence of chronic diseases—particularly of the lungs—remained unchanged in preterm infants (5). The incidence of BPD is about 40-60% of all VLWB preterm neonates (<1500 g weight at birth, VLBW=very low birth weight infants) (6-8). Since there are about 55,000 VLWB preterm neonates per year in USA, about 27,500 infants will develop a BPD each year. Due to the chronic pulmonary damage the initial length of stay in the hospital is significantly prolonged. The subsequent costs for the therapy of infants with chronic lung disease are estimated to be about [symbol] 1 million for each affected preterm neonate (9).

It is more difficult to estimate the frequency of oxygen-induced pulmonary damages in adults. Acute respiratory distress syndrome (ARDS) consist of a variety of pulmonary diseases that lead to severe pulmonary changes requiring at least some ventilatory support. This syndrome is sometimes also called adult respiratory distress syndrome, although it can occur in children. Typical histological signs of ARDS are fluid accumulation, inflammation and cellular damage in the lung. Due to these changes, systemic oxygenation is deteriorated and thus, supplemental oxygen is required. However, ARDS is partially triggered by an oxygen-associated toxicity (10). Due to the great variety of diseases that are associated with ARDS, great variations exist regarding the incidence of ARDS. In 1972 there was an incidence of 75 per 100,000 adults. However, recent studies assume a frequency of about 15 per 100,000 adults (11).

A major factor in lung vascular development is vascular endothelial growth factor (VEGF), which had first been described as a growth and survival factor of the vascular endothelium and hematopoetic stem cells (12). However, VEGF is found in a variety of tissues. Mice that lack the VEGF protein or one of the VEGF receptors (VEGF-R) are not viable (13). As of now four different human isoforms of VEGF and three different VEGF receptors have been described (14), wherein Flt-1 (VEGF-R1) and Flk/KDR (VERGF-R2) are receptor tyrosine kinases.

In the mature human lung VEGF is primarily found in alveolar type II cells (13), whereas in the premature human lungs of fetuses VEGF is located in the basal membrane of the airway epithelia. Thus, VEGF probably has an early impact on the vascularization of the airways (14). VEGF stimulates different signal transduction pathways by binding to its receptors, wherein also cross talk takes place. On the one hand, VEGF stimulates the proliferation and migration of blood vessels (VEGF-R1, VEGF-R2), and, on the other hand, VEGF increases the vascular permeability (VEGF-R3). The physiological response to VEGF depends on the dosage of VEGF and on the corresponding receptor distribution (15).

A paracrine mechanism for VEGF function has been postulated. Thus, VEGF can modulate the vascular endothelium, especially when produced by epithelial cells (16). In contrast to these initial findings it has been recently shown, that VEGF does not only influence endothelial cells, but it rather stimulates the proliferation and differentiation of epithelial cells in an autocrine manner (17). The expression of VEGF is variable and depends on various factors, especially the oxygen concentration. The oxygen dependent regulation of VEGF is mostly regulated by the hypoxia inducible factor (HIF) (18).

The pulmonary synthesis of VEGF and VEGF receptor is significantly stimulated by hypoxia (19, 20). During hyperoxia the VEGF production is suppressed. This suppression is considered to be (at least in part) responsible for the oxygen induced toxicity (21, 22). During hyperoxia the VEGF protein production rapidly decreases within the first 48 hours, whereas the concentration of the two receptors VEGF-R1 and VEGF-R2 decreases not until after 48 hours, probably secondarily due to the loss of endothelial cells (21). In a rat model, where the VEGF receptor was chemically blocked and, thus, the VEGF-dependent signal transduction was inhibited, the close relationship between oxidative stress and apoptosis has been demonstrated. Thereby, the oxidative stress led to an increased apoptosis and the subsequent development of a lung emphysema, but an increased apoptosis leads to an increased expression of oxidative stress markers as well (23).

Furthermore, VEGF plays an important role in the pathogenesis of broncho-pulmonary dysplasia of preterm neonates (BPD). Preterm newborns who develop a BPD have a lower VEGF concentration in the tracheal secret in the first days after birth compared to that of an appropriate control group without later BPD (24). In lung autopsy samples of preterm neonates, that had died of BPD, a lower expression of VEGF mRNA, VEGF protein and VEGF receptor in comparison to the control group was found (25). In the thickened alveolar septa of BPD patients the VEGF mRNA is reduced, while at the same time the angiogenic receptors Flt-1 (VEGF-R1) and TIE-2 are reduced as well. This suggests that the morphological changes that are typical for BPD are caused by the perturbed expression of VEGF, VEGF receptor and other angiogenic factors.

Moreover, VEGF mediates an anti-apoptotic effect. VEGF inhibits the TNFα-induced apoptosis in endothelial cells (26). VEGF mediates anti-apoptotic effects in endothelial cells via extrinsic (receptor-dependent) apoptotic cascades. Besides the influence on the intracellular signalling cascades the regulation of the cytoskeleton by VEGF becomes more important. Furthermore, VEGF induces the expression of the adhesion proteins fibronectin and integrin β3. The loss of their adhesion ability leads to an increased apoptosis and to cell death of endothelial cells (26). VEGF is not only a potent mitogen of the angiogenesis, it further has a protective effect on endothelial cells (15). VEGF induces the expression of the anti-apoptotic proteins Bcl-2 and A1 by activating phosphatidyl-inositol-3 kinase in vascular endothelial cells (27, 28). An antibody directed against VEGF-R1, that inhibits the receptor-mediated effect of VEGF, led to a high apoptosis index, an almost complete inhibition of growth and a high lethality of newborn mice. However, these changes are not found with juvenile and adult mice (29), which may probably be explained by the increased sensibility for VEGF of premature cells.

VEGF has already been used for the treatment of pulmonary hypertension. The U.S. Pat. No. 6,352,975 and the patent applications WO 00/013702 and WO 00/013703 disclose methods for the treatment of salt-sensitive hypertension. These methods involve administering VEGF in an amount effective to reduce the blood pressure of a patient suffering from salt-sensitive hypertension to a normal range. Thereby, the preferred method is that VEGF is co-administered with another angiogenic factor, or two or more VEGF are administered. Furthermore, the VEGF used can contain a heparin binding domain. WO 00/071716 further discloses disulfide-bonded dimeric VEGF useful for the treatment of hypertension.

The patent application WO 00/065043 uses recombinant defective adenovirus comprising a nucleic acid encoding an angiogenic factor, such as VEGF among others, for treating pulmonary arterial hypertension.

Further, the intra-ocular injection of VEGF is used to treat retinopathy of prematurity (ROP), which is initiated by hyperoxia-induced obliteration of newly formed blood vessels in the retina of premature newborns (30).

The patent application WO 02/086497 discloses the use of hypoxia inducible factor 2α (HIF-2α) or VEGF for the treatment of pulmonary hypertension and neonatal respiratory distress syndrome (nRDS). Thus, the patients are preterm infants with an immature lung. The immaturity is the reason for a surfactant deficiency and the subsequent respiratory distress. The treatment is carried out either by an intra-amniotical administration to unborn fetuses (i.e. prior to delivery) or an intra-tracheal administration after birth. The proposed effect of the VEGF is the improvement of surfactant production that is supposed to protect the preterm newborns against nRDS. Subsequently, the need for mechanical ventilation will be reduced. However, the proposed treatment is limited to one group of patients which have a surfactant deficiency due to their immature lungs. However, NRDS is already been treated successfully by two different strategies (since more than 10 years): the prenatal induction of lung maturation and the postnatal surfactant substitution. Thus, a replacement of these established therapies is unlikely.

There is a need for a pharmaceutical to treat and furthermore prevent lung injuries, especially chronic damages, in patients of all ages, where lung injury is related to a deregulation of the oxygen provision and is especially caused by high concentrations of inspired oxygen.

The objective has been solved according to the present invention by the use of VEGF for the manufacture of a medicament for the treatment and/or prevention of pulmonary diseases or conditions that are related to high concentrations of inspired oxygen. High concentrations of oxygen are considered as oxygen concentrations higher than in atmospheric air (fraction of inspired oxygen above 0.21).

The term “vascular endothelial growth factor” or “VEGF” as used herein refers to any naturally occurring (native) forms of a VEGF polypeptide (also known as “vascular permeability factor” or “VPF”) from any animal species, including humans and other mammalian species, such as murine, bovine, equine, porcine, ovine, canine, or feline, and functional derivatives thereof. “Native human VEGF” consists of two polypeptide chains generally occurring as homodimers. Each monomer occurs as one of five known isoforms, consisting of 121, 145, 165, 189, and 206 amino acid residues in length. These isoforms will be hereinafter referred to as hVEGF.sub.121, hVEGF.sub.145, hVEGF.sub.165, hVEGF.sub.189, and hVEGF.sub.206, respectively. Similarly to the human VEGF, “native murine VEGF” and “native bovine VEGF” are also known to exist in several isoforms, 120, 164, and 188 amino acids in length, usually occurring as homodimers. With the exception of hVEGF.sub.121, all native human VEGF polypeptides are basic, heparin-binding molecules. hVEGF.sub.121 is a weakly acidic polypeptide that does not bind to heparin. These and similar native forms, whether known or hereinafter discovered are all included in the definition of “native VEGF” or “native sequence VEGF”, regardless of their mode a preparation, whether isolated from nature, synthesized, produced by methods of recombinant DNA technology, or any combination of these and other techniques. The term “vascular endothelial growth factor” or “VEGF” includes VEGF polypeptides in monomeric, homodimeric and heterodimeric forms. The definition of “VEGF” also includes a 110 amino acids long human VEGF species (hVEGF.sub.110), and its homologues in other mammalian species, such as murine, bovine, equine, porcine, ovine, canine, or feline, and functional derivatives thereof. In addition, the term “VEGF” covers chimeric, dimeric proteins, in which a portion of the primary amino acid structure corresponds to a portion of either the A-chain subunit or the B-chain subunit of platelet-derived growth factor, add a portion of the primary amino acid structure corresponds to a portion of vascular endothelial growth factor. In a particular embodiment, a chimeric molecule is provided consisting of one chain comprising at least a portion of the A- or B-chain subunit of a platelet-derived growth factor, disulfide linked to a second chain comprising at least a portion of a VEGF molecule. More details of such dimers are provided, for example, in U.S. Pat. Nos. 5,194,596 and 5,219,739 and in European Patent EP-B 0 484 401, the disclosures of which are hereby expressly incorporated by reference. The nucleotide and amino acid sequences of hVEGF.sub.121 and bovine VEGF.sub.120 are disclosed, for example, in U.S. Pat. Nos. 5,194,596 and 5,219,739, and in EP 0 484 401. hVEGF.sub.145 is described in PCT Publication No. WO 98/10071; hVEGF.sub.165 is described in U.S. Pat. No. 5,332,671; hVEGF.sub.189 is described in U.S. Pat. No. 5,240,848; and hVEGF.sub.206 is described in Houck et al. Mol. Endocrinol. 5:1806-1814 (1991). Other VEGF polypeptides and polynucleotides have been described, including, for example, zvegf2 (PCT Publication No. WO 98/24811), and VRP (PCT Publication No. WO 97/09427), and are also encompassed by the term VEGF. For the disclosure of the nucleotide and amino acid sequences of various human VEGF isoforms see also Leung et al., Science 246:1306-1309 (1989); Keck et al., Science 246:1309-1312 (1989); Tisher et al., J. Biol. Chem. 266:11947-11954 (1991); EP 0 370 989; and PCT publication WO 98/10071. For further review, see also Klagsbum and D'Amore, Cytokine and Growth Factor Reviews 7:259-170 (1996).

The term “VEGF” encompasses a polypeptide having an amino acid sequence substantially homologous to one or more of the above-mentioned native VEGF polypeptides, and which retains a biological activity associated with VEGF. An amino acid sequence is considered to be “substantially homologous” herein if the level of amino acid sequence homology is at least about 50%, preferably at least about 80%, more preferably at least about 90%, most preferably, at least about 95%, compared with the native VEGF protein in question.

Also included within the scope of “VEGF” herein are biologically active fragments thereof, as well as N-terminally or C-terminally extended versions thereof or analogs thereof substituting and/or deleting or inserting one or more amino acid residues which retain qualitatively the biological activities of the protein described herein.

The term “VEGF” specifically includes homodimeric and heterodimeric forms of the VEGF molecule, in which the dimer is formed via interchain disulfide bonds between two subunits. Homodimers may have both of their subunits unglycosylated or glycosylated, while in heterodimers, one subunit may be glycosylated and the other unglycosylated. The term “VEGF” specifically includes not only amino acid sequence variants but also glycosylation variants of the native VEGF molecules.

In addition, the term “VEGF” covers chimeric, dimeric proteins, in which a portion of the primary amino acid structure corresponds to a portion of either the A-chain subunit or the B-chain subunit of platelet-derived growth factor, add a portion of the primary amino acid structure corresponds to a portion of vascular endothelial growth factor.

In a further aspect of the present invention, the pulmonary diseases or conditions treated and/or prevented by the use of VEGF are associated with a deregulation of the oxygen provision.

In another preferred aspect, the pulmonary diseases or conditions treated and/or prevented by the use of VEGF require oxygen supplementation. Oxygen supplementation can be performed by oxygen given by hood or nasal cannula, continuous positive airway pressure (CPAP), mechanical ventilation.

In a preferred aspect, the pulmonary diseases or conditions treated and/or prevented by the use of VEGF are caused by hyperoxia.

In another preferred aspect, the pulmonary disease or condition treated and/or prevented by the use of VEGF is caused by a “relative hyperoxia”, that means an oxygen concentration that is higher than under normal physiological conditions.

Hyperoxia changes the metabolism of pulmonary type II cells. In addition to changes in surfactant composition (31), lipid peroxidation (32), immunological parameters (33) as well as surfactant lipid synthesis (34) pro-apoptotic changes have been found. These pro-apoptotic changes can be summarized as follows: hyperoxia increases TNFα—an inducer of apoptotic changes—in type II cells and in the alveolar lavage. Furthermore, the expression of the TNFα receptor 1 is increased in type II cells. By activating the TNFα receptor 1 the enzyme activity of the caspases 8 and 3 are induced, which leads to an increased apoptosis (35). Following hyperoxic damage an increased caspase 8 and 3 activity is found in type II cells.

VEGF stimulates the proliferation of epithelial cells in the lungs as well as mediates an anti-apoptotic effect, while hyperoxia leads to an inhibition of the VEGF secretion of pulmonary type II cells.

Different pulmonary diseases, that are mainly caused by a deficiency (nRDS) or an inhibition of the pulmonary surfactant, lead to a respiratory insufficiency and, thus, to the need of oxygen supplementation, ventilatory support or mechanical ventilation. The high concentration of inspired oxygen induces an apoptotic cascade in the lung, which causes chronic changes in the lung. The application of VEGF interrupts the cascade so that no damage occurs. This effect is independent on the influence on the pulmonary surfactant system or the morphological maturation of the lung, respectively. The use is furthermore not limited to the neonatal period.

In a preferred aspect, the VEGF is applied intra-tracheal. The application of VEGF, intra-tracheal or by inhaling, is preferred, because systemic side effects using this way of application are very unlikely to occur. In general, VEGF is applied in the same manner as it is already known and used for surfactant application. Thus, a person skilled in the art would be able to apply this knowledge for the VEGF application.

The preferred VEGF used is a human VEGF. The VEGF can be recombinant human VEGF and appropriate fragments thereof, VEGF analogues as well as appropriate fragments thereof. In one embodiment, the invention provides the use of VEGF₁₆₅ for the manufacture of a medicament, however, other VEGF forms or fragments thereof can be used equally. VEGF used can contain a heparin binding domain. Furthermore it can be disulfide-bonded dimeric VEGF.

The expression of VEGF and VEGF receptor is reduced in type II cells of hyperoxia-treated animals. After only one single intra-tracheal VEGF application, which was applied before the start of an 48 hour period of hyperoxia, the VEGF protein and mRNA-concentration in the lung was significantly higher than in the hyperoxia group, and comparable to the normoxic control group. The expression of VEGF receptor (protein and mRNA) in hyperoxia-damaged type II cells was significantly decreased. After intra-tracheal application of VEGF almost no changes, compared to normoxic control group, were found despite of the exposure to high concentrations of inspired oxygen for 48 hours.

Furthermore, the expression of TNFα and TNFα receptor 1 (TNFα-R1) is increased during hyperoxia. TNFα is an important inducer of apoptosis. After a single intra-tracheal application of VEGF prior to the start of the hyperoxic exposure, the TNFα protein and mRNA concentration in type II cells is significantly lower when compared with type II cells from hyperoxia-damaged animals without VEGF administration. At the same time the TNFα-R1 concentration was also reduced in type II cells of the hyperoxia-treated animals with prior intra-tracheal application of VEGF.

Besides the changes in the TNFα and TNFα-R1, changes in the cytoskeleton are found in type II cells of hyperoxic damaged animals. Hyperoxia causes an abolishment of the directed structures of the cytoskeleton. A destabilization of the cytoskeleton is associated with an increased apoptosis and subsequent cell death. After an intra-tracheal administration of VEGF prior to the hyperoxic exposure no derangement of the cytoskeleton in type II cells was observed.

Alterations in cytoskeleton are associated with an activation of NF-κB. The nuclear factor-κB (NF-κB) is a key factor for the control of different cellular pathways and is involved in inflammation and proliferation. Furthermore, the activation of NF-κB represents one of the most effective anti-apoptotic survival pathways in mammals (39). The inactive NFκB is located in the cytoplasm. NF-κB RelA (p65) and NF-κB1 (p50) subunits are sequestered in an inactive complex by binding to its inhibitory proteins of the IκB family. Various stimuli cause a degradation of IκBα, thereafter p50/p65 can enter the nucleus and induce various cellular processes. Thus, a degradation of cytoplasmatic IκBα concentration is representative for an activation of NF-κB. Intra-tracheal substitution of VEGF compensates for the hyperoxia-induced deficiency of endogenous VEGF, minimizes the hyperoxia-induced damage and, therefore, prevents the development of an acute or a chronic lung disease, in particular.

The term “medicament for the treatment and/or prevention” relates to a composition comprising VEGF as described above and a pharmaceutically acceptable carrier or excipient to treat or prevent diseases as indicated above. The compound or a pharmaceutically acceptable salt thereof may be administered. The active compound may be administered alone or preferably formulated as a pharmaceutical composition.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The most preferred carrier is saline, sterile aqua or glucose solution. Furthermore, inclusion into lipsomes can be utilized if desired.

Pharmaceutically acceptable salts are non-toxic at the concentration at which they are administered. Pharmaceutically acceptable salts include acid addition salts such as those containing sulfate, hydrochloride, phosphate, sulfonate, sulfamate, sulfate, acetate, citrate, lactate, tartrate, methanesulfonate, ethanesulfonate, benzenesulfonate, p-toluenesulfonate, cyclolexylsulfonate, cyclohexylsulfamate and quinate. Pharmaceutically acceptable salts can be obtained from acids such as hydrochloric acid, sulfuric acid, phosphoric acid, sulfonic acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfonic acid, cyclohexylsulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

The amount effective to treat or prevent the disorders herein described depends on the usual factors, such as nature and severity of the disorders and the weight of the mammal. However, a unit dose will preferably contain 0.01 to 50 mg, for example 0.01 to 10 mg, or 0.05 to 2 mg of the identified compound or a pharmaceutically acceptable salt thereof. Unit doses will normally be administered once or more than once a day, for example 2, 3 or 4 times a day, more usually 1 to 3 times a day, such that the total daily dose is normally in the range of 0.0001 to 1 mg/kg; thus a suitable daily dose for a 70 kg adult is 0.01 to 50 mg, for example 0.01 to 10 mg or more usually 0.05 to 10 mg. It is preferred that the compound or a pharmaceutically acceptable salt thereof is administered in the from of a unit-dose composition.

Patients which receive oxygen supplementation, i.e. which require high concentrations of inspired oxygen, e.g. during mechanical ventilation, can be treated with the medicament according to the invention for the prevention of acute damages of the lungs as well as for the prevention of chronic damages of the lungs.

The change in the VEGF concentration is part of the pathogenesis of hyperoxia-induced pulmonary damage. The BPD of preterm infants as well as the ARDS or other chronic lung diseases of adults are based (among other causes) on an oxygen-associated toxicity, which is mediated by a VEGF deficiency. Substitution with VEGF can compensate for that deficiency and so minimize the hyperoxia-induced damages.

The patients that can be treated with a medicament according to the invention can be patients of all ages, i.e. the patients are selected from the group comprising adults, infants, neonates or preterm infants.

The pulmonary diseases preferably treated or prevented by the medicament according to the invention are selected from the group comprising broncho-pulmonary dysplasia (BPD) of premature infants, respiratory distress syndrome, acute respiratory distress syndrome (ARDS), neonatal respiratory distress syndrome (NRDS) and/or other diseases or conditions that are associated with high concentrations of inspired oxygen.

Acute respiratory distress syndrome (ARDS), represents a severe form of respiratory insufficiency that often occurs in patients with lung disease of various origin. Beside mechanical ventilation and treatment of the underlying disease, a therapy with high concentrations of inspired oxygen is vital to improve systemic oxygenation.

The respiratory distress syndrome (RDS) is found in preterm infants and is caused by surfactant deficiency due to the immaturity of the lung. The RDS does almost exclusively occur in newborns born before 37 week of gestation; the more premature the greater the risk of developing a RDS. Infants with RDS show symptoms of respiratory insufficiency. The subsequent deterioration in systemic oxygenation can cause multiple organ failure and death. Treatment include intra-tracheal administration of pulmonary surfactant after birth and high concentrations of inspired oxygen with or without the need for mechanical ventilation.

Broncho-pulmonary dysplasia (BPD) is a chronic lung disease of the preterm infant, that causes persistent respiratory distress and rarefaction or fibrosis of alveolar structures. Prematurity is the predisposing factor for subsequent development of BPD. In premature lungs, either mechanical damage (due to mechanical ventilation), inflammation or oxygen toxicity lead to the subsequent development of BPD.

The medicament can further comprise surfactant. Surfactant is a mixture of phospholipids and surfactant-associated proteins (SP-A to SP-D), which lowers surface tension at the air-water interface and thereby prevents alveolar collapse and respiratory failure. Surfactant is used to treat severe respiratory insufficiency which requires oxygen supplementation. Thereby, surfactant is applied intra-tracheal. A combination of VEGF and surfactant is preferred and can be used as a medicament for the treatment and/or prevention of pulmonary diseases or conditions that are related to high concentration of inspired oxygen. A wide variety of surfactant products were studied in clinical trials and are commercially available, such as synthetic surfactants and natural surfactant extracts. Natural surfactant extracts are derived from animal sources. Currently used synthetic surfactants are complex combinations of dipalmitoylphosphatidylcholine and other phospholipids, neutral lipids, lipoprotein, or alcohols. Components of synthetic surfactants are not directly obtained from the extraction of surfactant from animal lung. New generations of synthetic surfactant do also contain analogs of either surfactant protein B or C or simplified peptides. Either surfactant can be potentially used in the medicament according to the invention. Examples for artificial surfactants are Exosurf Neonatal (a synthetic surfactant composed of dipalmitoylphosphatidylcholine, hexadecanol and tyloxapol), Lucinactant (Surfaxin®, Discovery Laboratories, Inc., Warrington, Pathologie., USA) (a mixture of dipalmitoylphosphatidylcholine, palmitoyloleoylphosphatidylglycerol, palmitic acid and KL₄) or Lusupultide (Venticute®, Altana Pharma, Konstanz, Germany) (a mixture of phospholipids and recombinant SP-C). Preferably natural surfactants will be used, since the clinical efficacy is superior to artificial surfactants. Preferred natural surfactants will be selected from the group comprising Curosurf®, Survanta®, or Alveofact®, however, other preparations of surfactant could be used equally. Curosurf® (Chiesi company, Italy) is a lipid extract from whole minced porcine lung tissue. Survanta® (Abbott GmbH, Wiesbaden, Germany) is prepared from minced bovine lung extract with added DPPC, triacylglycerol and palmitic acid. Alveofact® (Boehringer Ingelheim Pharma KG, Ingelheim, Germany) is produced by lipid extraction from bovine lung lavage.

The medicament can be administered in combination with perflurocarbons (PFC). PFC are substances that are investigated for therapy of acute respiratory failure. Due to intratracheal application of PFC systemic oxygenation is improved. Combination of drugs with PFC allow a homogenous distribution of the drug (such as surfactant (41)) in the lung. Combination of VEGF and PFC will improve the efficacy of VEGF action even in atelectatic lung areas.

Administration “in combination with” one or more further therapeutic agents includes simultaneous (concurrent) and consecutive administration in any order.

Furthermore, an inhalation of PFC has been shown to be effective (42, 43). Combination of VEGF and PFC and a subsequent inhalation allows an administration in non-ventilated patients.

The medicament according to the invention is preferably used in an intensive care therapy. The intensive care therapy is a specific utilization that will be very valuable in reducing the costs that are caused by the oxygen associated pulmonary damage.

For a better understanding of the present invention, the following drawings and examples further illustrate various embodiments of the invention. The drawings and examples are not intended to be limiting in any way.

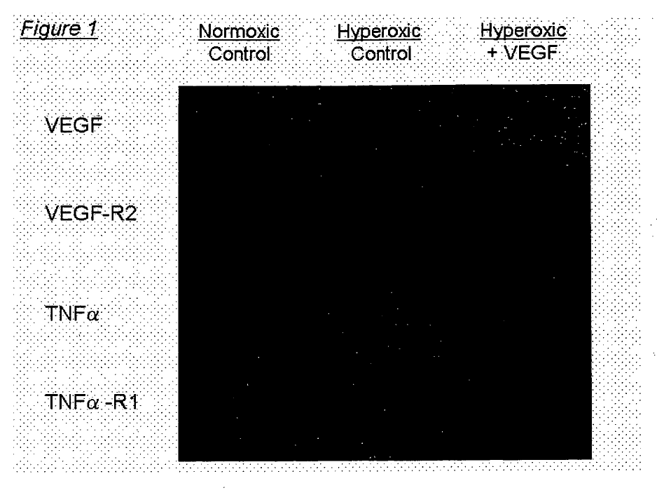

FIG. 1: Changes in VEGF (first row), VEGF-R2 (second row), TNFα (third row) and TNFα-R1 (fourth row) in lungs from rat that were exposed to sublethal hyperoxia (second column) or received VEGF intra-tracheally prior to the exposure to sublethal hyperoxia (last column). For comparison, immunohistographs of lungs from healthy, normoxic animals (normoxic control) are shown in the first column.

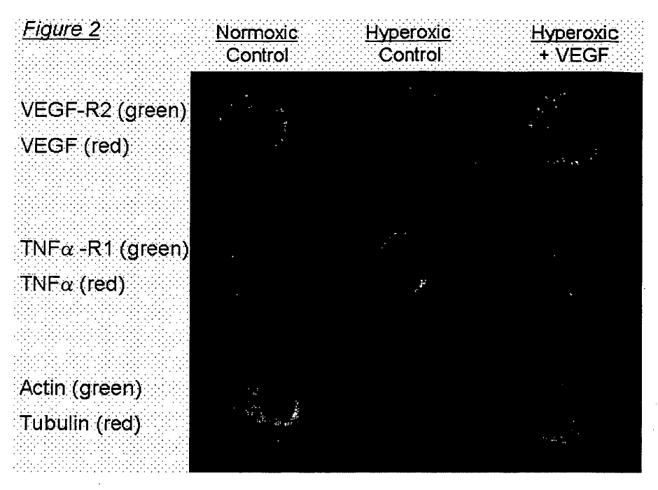

FIG. 2: Changes in VEGF-R2 and VEGF (first row), TNFα-R1 and TNFα (second row), actin and tubulin (last row) in type II pneumocytes from rats that were exposed to sublethal hyperoxia (hyperoxic control—second column) or received VEGF intratracheally prior to the exposure to sublethal hyperoxia (hyperoxic+VEGF—last column). For comparison, control type II pneumocytes are shown in the first column (normoxic control).

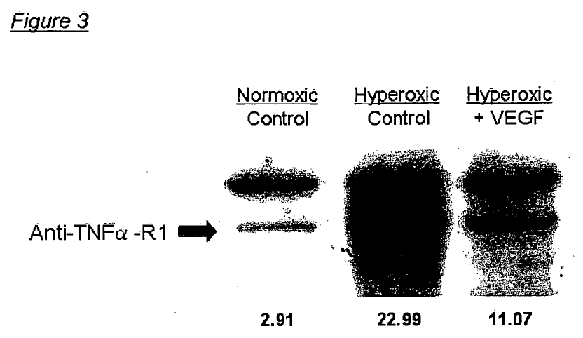

FIG. 3: Protein concentration of TNFα-R1 in the membrane fraction of type II pneumocytes analyzed by western blotting. Type II cells were obtained from rats that were exposed to normoxic conditions (normoxic control—first column), sublethal hyperoxia (hyperoxic control—second column) or received VEGF intra-tracheally prior to the exposure to sublethal hyperoxia (hyperoxic+VEGF—last column). Values of densitometric quantification are given below the individual blot.

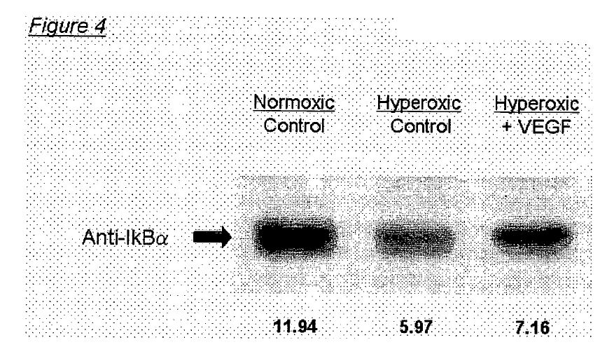

FIG. 4: Protein concentration of IκBα in the cytoplasmatic extracts of type II pneumocytes analyzed by western blotting. Type II cells were obtained from rats that were exposed to normoxic conditions (normoxic control—first column), sublethal hyperoxia (hyperoxic control—second column) or received VEGF intra-tracheally prior to the exposure to sublethal hyperoxia (hyperoxic+VEGF—last column). Values of densitometric quantification are given below the individual blot.

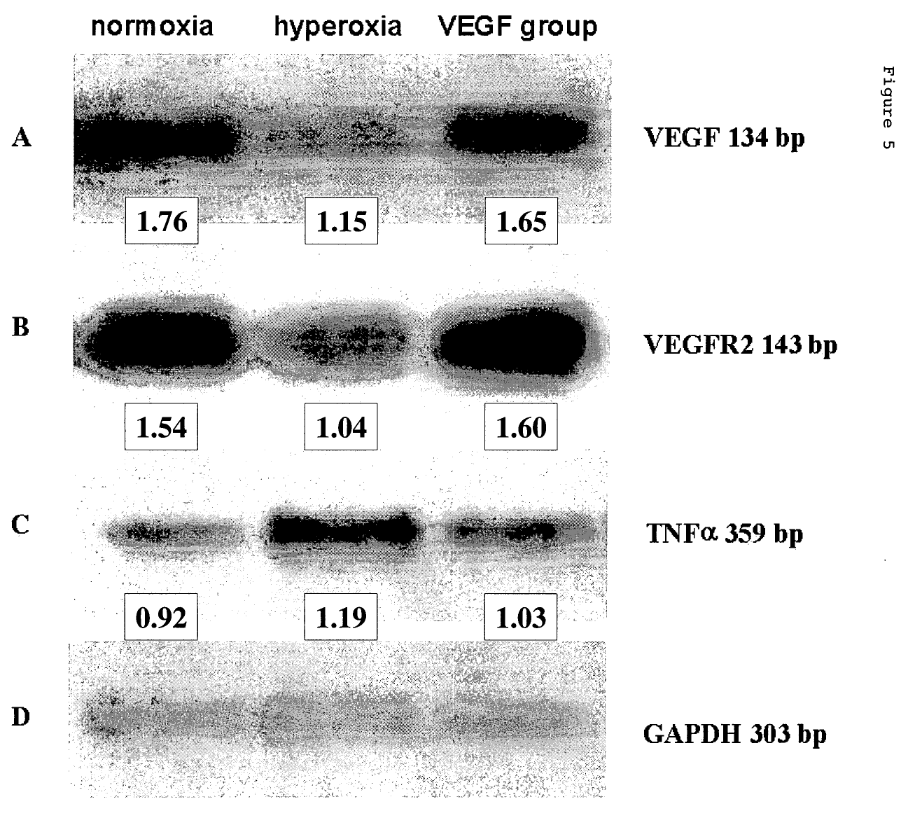

FIG. 5: Changes in VEGF (A-first row), VEGF-R2 (B-second row) and TNFα (C-third row) mRNA expression, isolated from type II pneumocytes obtained from rat that were exposed to sublethal hyperoxia (second column) or received VEGF intra-tracheally prior to the exposure to sublethal hyperoxia (last column). For comparison, mRNA expression of type II pneumocytes from healthy, normoxic animals (normoxic control) are shown in the first column. Expression of GAPDH, as shown in the last column (D), is homogenous in all three groups. Values of densitometric quantification (given as a ratio of the individual signal and GAPDH) are presented below the individual blot.

### EXAMPLE 1

### Sublethal Hyperoxia

For studying hyperoxia-induced pulmonary changes an established sublethal animal model was used (31). Adult Wistar rats (body weight approximately 120 g), each placed in an individual plastic chamber, which is ventilated with oxygen (flow 1 L/min), were continuously gassed with 100% oxygen for 48 hours. The oxygen-measurement directly beside the head of the rats revealed that the oxygen concentration was 75 to 80% oxygen. Water and food was available ad libitum. Preparation of the bronchoalveolar lavage, alveolar macrophages and type II cells (Tllcells) were carried out as previously described (36).

### EXAMPLE 2

### Intra-tracheal Application of Human Recombinant VEGF

Wistar rats were anesthesized by inhalation of ether. For intra-tracheal VEGF application the rates were intubated with a plastic cannula. The rats obtained intra-tracheal 200 μl 0.9% PBS (control) or 5 μg of recombinant human VEGF (PeproTech EC Ltd., London) in 200 μl PBS per animal, respectively. After extubation and a short recovery period in normal air, the rats which obtained VEGF or PBS were kept at hyperoxic condition (see Example 1 above) for 48 h, one control rat was kept under normoxic conditions. T_{II}cells were isolated and further studies were performed.

### EXAMPLE 3

### Immunohistochemistry

Immunohistochemistry and microscopy were carried out on entire lungs that were fixated by intravenous formaledehyd perfusion or on fixated isolated type II cells as previously described (36). The following antibodies were used: Rabbit polyclonal anti-rat TNFα antibody from BioSource Europe (Nivelles, Belgium), rabbit polyclonal anti-rat TNFα-R1 antibody raised against a recombinant peptide (amino acids 30-301) including the extracellular domain of TNFα-R1 (Santa Cruz Biotechnology, Heidelberg, Germany), mouse monoclonal VEGF antibody corresponding to amino acids 1-140 of VEGF (Santa Cruz Biotechnology, Heidelberg, Germany). To study the cytoskeleton, Alexa 488-conjugated phalloidin was used as a specific fluorescent probe for F-actin and a monoclonal antibody anti-β-tubulin (Molecular Probes, Eugene, Oreg. USA) was used to label β-tubulin. Secondary antibodies conjugated with Alexa 499 and Alexa 594 were from Molecular Probes Europe BV (Leiden, Netherlands). Lung tissue sections were labeled with specific antibodies directed against TNFα-R1, TNFα, VEGF and VEGF-R2.

For double staining, the labeled preparations were analyzed using a confocal laser scanning microscope (CLSM, Leica Microsystems AG, Wetzlar, Germany), equipped with an argon/krypton laser. Images were taken using a 40×NA 1.3 oil objective to fluorescent excitation and emission spectra for Alexa 488 (Excitation 490 nm, emission 520 nm) and for Alexa 594 (excitation 541 nm, emission 572 nm). With the dual-channel system of the confocal microscope, dual-emission (535/590 nm) images were recorded simultaneously with a scanning speed at 16 s/frame (512 lines). Images were obtained and processed using TCS NT Version 1.S.451 (Leica Microsystems AG, Wetzlar, Germany). As control the tissue slides were incubated with the Alexa-labeled secondary antibodies without prior incubation in presence of the specific first antibody. No unspecific binding of the secondary antibodies occurred.

### EXAMPLE 4

### Western Blot Analysis of TNFα-R1

Stored alveolar type II cells were resuspended in homogenization buffer (20 mM Tris/HCl, pH 7.5, 1 mM EDTA, 1 mM EGTA, 250 mM saccharose, 1 mM DTT, 1 mM PMSF, 10 μg/ml aprotinin, 10 μg/ml leupeptin, and a Boehringer protease inhibitor tablet/10 ml) according to Gobran and Rooney (37) and the cells were sonicated (2×20 s; Sonoplus HD60, Bandelin Electronics, Berlin, Germany). The separated fraction was collected by centrifugation at 100,000×g. All steps were carried out at 0-4° C. The supernatants were subjected to electrophoresis and immunoblotting for analysis of TNFα-R1. In brief, proteins were separated by standard SDS-PAGE on 8% gels and electro-blotted onto nitrocellulose. For visualizing the protein-bands a rabbit polyclonal anti-TNFα-R1 antibody (Santa Cruz Biotechnology, Heidelberg, Germany) and a goat anti-rabbit peroxidase-conjugated secondary antibody was used. The peroxidase/chemiluminescence bands on Kodak X-OMAT films were quantified by scanning using a densitometer with automatic calibration (Image Master DTS; Pharmacia, Uppsala, Sweden) and GS-710 Imaging Densitometer (Bio-Rad, Hercules, Calif.).

### EXAMPLE 5

### Western Blot Analysis of IκBα

Cytoplasmatic extracts of isolated type II cells were made according to the methods described previously by Altavilla et al. (40). The protein content of cytoplasmic extracts was determined by Bradford assay using bovines serum albumin (BSA) as a standard method (Bio-Rad Laboratories protein assay kit, Richmond, USA). IκBα protein was assayed in cytoplasmatic extracts of type II cells by Western blotting. Cytosolic proteins (40 μg) from each sample were mixed with 2× sodium dodecyl sulfate (SDS) sample buffer, heated at 95° C. for 5 minutes, and separated by 12.5% SDS-polyacrylamide gels. The separated proteins were blotted onto nitrocellulose membrane. Non-specific binding sites were subsequently blocked with 5% nonfat dry milk in TBS-0.05% Tween. Mouse monoclonal IgG1 anti-I-κBc (H-4) (Santa Cruz Biotechnology, Heidelberg, Germany) antibodies were used for primary detection. Peroxidase-conjugated anti-mouse IgG antibodies (Dianova, Hamburg, Germany) was employed for secondary detection. The peroxidase/chemiluminescence-produced bands were visualized on film by enhanced chemiluminescence (ECL) (Amersham Biosciences, Inc, Piscataway, N.J.). The bands on films were quantified by scanning densitometry.

### EXAMPLE 6

### Determination of TNFαConcentration

To determine the TNFα content in type II cells, macrophages, plasma or cell-free bronchoalveolar lavage, a commercially available ELISA kit from Biosource (Ratingen, Germany) was used.

### EXAMPLE 7

### Sublethal Hyperoxia Induces a Pro-apoptotic Situation in Type II Pneumocytes

In the first set of experiments the effect of sublethal hyperoxia upon various apoptotic parameters in the type II pneumocytes was studied (38).

In summary, it was shown, that hyperoxia induces an increase in TNFα in type II cells, in bronchoalveolar lavage and serum. The rise in TNFα is associated with an increase in caspase 8 and 3 and thus, a pro-apoptotic situation is initiated. These hyperoxia-induced apoptotic responses are suppressed by an inhibition of the TNFα-receptor activation. Thus, TNFα represents the key signal for oxygen-induced pulmonary damage.

### EXAMPLE 8

### Intra-tracheal VEGF Administration Improves VEGF and VEGF-R2 Concentration in Lungs and Isolated Type II Cells

Rats were treated with VEGF intra-tracheally, prior to exposure to sublethal hyperoxia. Analysis of VEGF and VEGF receptor expression revealed a significant reduction of VEGF and VEGF-R2 in entire lungs of rats exposed to hyperoxia. After application of VEGF not only the hyperoxia-induced reduction of VEGF but also the reduced expression of VEGF-R2 was prevented. VEGF and VEGF-R2 were comparable to control animals under normoxic conditions (FIG. 1). The results were even more prominent if isolated type II pneumocytes were analyzed (FIG. 2).

### EXAMPLE 9

### Intratracheal VEGF Administration Affects TNFα and TNFα-R1 Concentration in Lungs and Isolated Type II Cells

Since the data of Example 6 above suggest, that hyperoxia-induced TNFα increase represents the key signal for oxygen induced pulmonary damage, the effect of VEGF application upon TNFα and TNFα-receptor on type II cells was studied.

An increase of TNFα and TNFα-R1 after hyperoxic exposure was observed. After VEGF application TNFα and TNFα-R1 concentration were significantly reduced when compared with hyperoxia control cells (FIG. 1). The results found in entire lungs were supported by immunostaining of isolated type II cells (FIG. 2). The immunhistochemical data were verified by western blot analysis (FIG. 3).

Furthermore, the concentration of TNFα in bronchoalveolar lavage, type II cells and serum is analyzed by ELISA.

### EXAMPLE 10

### Intratracheal VEGF Administration Prevents Changes in Cytoskeleton

The effect of hyperoxia and VEGF upon the cytoskeleton was studied. Hyperoxia caused a significant alteration and reduction in actin and an increase in tubulin. Intratracheal application of VEGF prior to hyperoxic exposure prevented the changes in cytoskeleton. Actin and tubulin staining were similar to control cells under normoxic conditions (FIG. 2).

### EXAMPLE 11

### Intratracheal VEGF Administration and IκBα Concentration in Isolated Type II Cells

Changes in cytoskeleton cause an activation of NFκB activity, with a degradation of cytoplasmatic IκBα. According to example 10, intratracheal VEGF application prevents changes in cytoskeleton. In the investigated dosage of intratracheal VEGF, the hyperoxia induced degradation of IκBα was prevented (FIG. 4).

### EXAMPLE 12

### Reverse Transcriptase-PCR (RT-PCR)

Semi-quantitative polymerase chain reaction was used to assess VEGF, VEGFR2 and TNFα mRNA expression in isolated rat type II cells. Total cellular RNA was extracted from 5×10⁶ isolated ATII cells using the RNeasy Mini Kit (QIAGEN GmbH Hilden, Germany), transcribed into cDNA by use of AMV reverse transcriptase (Invitrogen GmbH, Karlsruhe, Germany) and random hexamers (Promega GmbH, Mannheim, Germany) according to the manufacturer's recommendations. The cDNA was amplified by PCR in a microprocessor-driven thermal cycler (Fa. Landgraf, Hannover, Germany) as previously described using the following forward (F) and reverse (R) oligonucleotides (BIO TeZ Berlin-Buch GmbH, Berlin, Germany):

GAPDH rat (GenBank BC059110; product size 303 bp):
F 5′-CAG TGC CAG CCT CTG CTC AT, R 5′-ATA CTC AGC
ACC AGC ATC AT.
VEGF rat (GenBank NM_031836; product size 134 bp):
F 5′-TGC ACT GGA CCC TGG CTT TAC, R 5′-CGG CAG TAG
CTT CGC TGG TAG.
VEGFR2 rat
(GenBank NM_013062; product size 143 bp):
F 5′-TAG CTG TCG CTC TGT GGT TCT G, R 5′-CCT GCA
AGT AAT CTG AAG GGT T.
TNFα rat (GenBank NM_012675; product size 359 bp):
F 5′-GGG GCC ACC ACG CTC TTC TGT, R 5′-GCA AAT CGG
CTG ACG GTG TGG.

Products were stained with ethidium bromide and electrophoresed through a 1.5% agarose gel. After transferring the PCR products to nylon membranes (Amersham, Braunschweig, Germany) by capillary blotting using 20×SSC as blotting buffer, the membranes were fixed by UV light and the incorporated digoxigenin-UTP was visualised by staining with anti-digoxigenin antibody conjugated to alkaline phosphatase (Roche Diagnostics GmbH, Mannheim, Germany), (18). Luminescence of the substrate (Lumigen™ PPD) was documented by short exposure to X-ray film (Kodak AG, Stuttgart, Germany). Densitometric calculations of digital film images were performed with the analysis program Scion Image, Version Beta 4.0.2 (Scion Corporation, Frederick, Md.).

### EXAMPLE 13

### Intra-tracheal VEGF Administration Improves VEGF and VEGF-R2 mRNA Expression in Isolated Type II Cells

Rats were treated with VEGF intra-tracheally, prior to exposure to sublethal hyperoxia. Analysis of VEGF and VEGF receptor mRNA expression revealed a significant reduction of VEGF and VEGF-R2 mRNA in type II pneumocytes obtained from lungs of rats exposed to hyperoxia. After application of VEGF not only the hyperoxia-induced reduction of VEGF mRNA but also the reduced expression of VEGF-R2 mRNA was prevented. VEGF and VEGF-R2 mRNA expression were comparable to control animals under nornoxic conditions (FIG. 5).

### EXAMPLE 14

### Intratracheal VEGF Administration Affects TNFα mRNA Expression in Isolated Type II Cells

An increase of TNFα mRNA after hyperoxic exposure was observed. After VEGF application TNFα mRNA expression was significantly reduced when compared with hyperoxia cells (FIG. 5).

### REFERENCES

1. Wispe J and Robert R: Molecular basis of pulmonary oxygen toxicity. Clin Perinatol 1987;14:651-666.

2. Philip A G: Oxygen plus pressure plus time: The etiology of bronchopulmonary dysplasia. Pediatrics 1975; 55:44-52.

3. Sinha P, Kolleck I, Volk H D, Schlame M, Rustow B. Vitamin E deficiency sensitizes alveolar type TI cells for apoptosis. Biochim Biophys Acta. 2002 Jun. 13;1583(1):91-8.

4. Northway W H, Rosan R C, Porter D Y: Pulmonary disease following respirator therapy of hyaline-membrane disease. N Engl J Med 1967; 276:357-368.

5. Manktelow B N, Draper E S, Annamalai S, Field D J: Factors affecting the incidence of chronic lung disease of prematurity in 1987, 1992, and 1997. Arch Dis Child Fetal Neonatal Ed 2001; 85:F33-F35.

6. Ogawa Y, Takasaki J, Itakura Y, Shimizu H: Epidemiology and classification of chronic lung disease. Ped Piilnonology 1997; Suppl.16:25-26.

7. Marshall D D, Kotelchuck M, Young T E, Bose C L, Kruyer L, O'Shea T M: Risk factors for chronic lung disease in the surfactant era: A north Carolina population-based study of very low birth weight infants. Pediatrics 1999; 104:1345-1350.

8. Lemons J A, Bauer C R, Oh W, Korones S B, Papile L A, Stoll B J, Verter J, Temprosa M, Wright L L, Ehrenkranz R A, Fanaroff A A, Stark A, Carlo W, Tyson J E, Donovan E F, Shankaran S, Stevenson D K: Very low birth weight outcomes of the national institute of child health and human development neonatal research network, january 1995 through december 1996. Pediatrics 2001; 107 el:1-8.

9. Greenough A, Alexander J, Burgess S, Chetcuti P A, Cox S, Lenney W, Turnbull F, Shaw N J, Woods A, Boorman J, Coles S, Turner J: Home oxygen status and rehospitalisation and primary care requirements of infants with chronic lung disease. Arch Dis Child 2002;86:40-4.

10. Lewis J F, Brackenbury A: Role of exogenous surfactant in acute lung injury. Crit Care Med 2003; 31:S324-328.

11. Atabai K, Matthay M A. The pulmonary physician in critical care 5: Acute lung injury and the acute respiratory distress syndrome: definitions and epidemiology. Thorax 2002;57:452-458.

12. Ferrara N and Davis-Smyth T: The biology of vascular endothelial growth factor. Endocr Rev 1997;18:4-25.

13. Gerber H P, Hillan K J, Ryan A M, Kowalski J, Keller G A, Rangell L, Wright B D, Radtke F, Aguet M, Ferrara N. VEGF is required for growth and survival in neonatal mice. Development 1999;126:1149-59.

14. Ferrara N, Gerber H P. The role of vascular endothelial growth factor in angiogenesis. Acta Haematol 2001; 106(4):148-56.

15. Becker P M, Verin A D, Booth M A, Liu F, Birukova A, Garcia J G. Differential regulation of diverse physiological responses to VEGF in pulmonary endothelial cells. Am J Physiol Lung Cell Mol Physiol 2001;281(6):L1500-11.

16. Shifren J L, Doldi N, Ferrara N, Mesiano S, Jaffe R B. In the human fetus, vascular endothelial growth factor is expressed in epithelial cells and myocytes, but not vascular endothelium: implications for mode of action. J Clin Endocrinol Metab 1994;79(1):316-22.

17. Brown K R, England K, Goss K L, et al. VEGF induces airway epithelial cell proliferation in human fetal lung in vitro. Am J Physiol Lulng Cell Mol Physiol 2001; 281:L1001-L1010.

18. Carmeliet P, Dor Y, Herbert J M, Fukumura D, Brusselmans K, Dewerchin M, Neeman M, Bono F, Abramovitch R, Maxwell P, Koch C J, Ratcliffe P, Moons L, Jain R K, Collen D, Keshert E, Keshet E. Role of HIF-1alpha in hypoxia-mediated apoptosis, cell proliferation and tumour angiogenesis. Nature 1998;394:485-90.

19. Christou H, Yoshida A, Arthur V, Morita T, Kourembanas S. Increased vascular endothelial growth factor production in the lungs of rats with hypoxia-induced pulmonary hypertension. Am J Respir Cell Mol Biol 1998; 18:768-76.

20. Compemolle V, Brusselmans K, Acker T, et al. Loss of HIF-2a and inhibition of VEGF impair fetal lung maturation, whereas treatment with VEGF prevents fatal respiratory distress in premature mice. Nature Medicine 2002; 8:702-710.

21. Klekamp J G, Jarzecka K, Perkett E A. Exposure to hyperoxia decreases the expression of vascular endothelial growth factor and its receptors in adult rat lungs. Am J Pathol 1999;154:823-31.

22. D'Angio C T, Maniscalco W M. The role of vascular growth factors in hyperoxia-induced injury to the developing lung. Front Biosci 2002;7:d1609-23. Review

23. Tuder R M, Zhen L, Cho C Y, Taraseviciene-Stewart L, Kasahara Y, Salvemini D, Voelkel N F, Flores S C. Oxidative stress and apoptosis interact and cause emphysema due to vascular endothelial growth factor receptor blockade. Am J Respir Cell Mol Biol 2003;29:88-97.

24. Lassus P, Ristimaki A, Ylikorkala O, Viinikka L, Andersson S. Vascular endothelial growth factor in human preterm lung. Am J Respir Crit Care Med 1999;159:1429-33.

25. Bhatt A J, Pryhuber G S, Huyck H, Watkins R H, Metlay L A, Maniscalco W M. Disrupted pulmonary vasculature and decreased vascular endothelial growth factor, Flt-1, and TIE-2 in human infants dying with bronchopulmonary dysplasia. Am J Respir Crit Care Med 2001;164:1971-80.

26. Spyridopoulos I, Brogi E, Kearney M, Sullivan A B, Cetrulo C, Isner J M, Losordo D W. Vascular endothelial growth factor inhibits endothelial cell apoptosis induced by tumor necrosis factor-alpha: balance between growth and death signals. J Mol Cell Cardiol. 1997;29:1321-30.

27. Alavi A, Hood J D, Frausto R, Stupack D G, Cheresh D A. Role of Raf in vascular protection from distinct apoptotic stimuli. Science 2003;301:94-6.

28. Gerber H P, McMurtrey A, Kowalski J, Yan M, Keyt B A, Dixit V, Ferrara N. Vascular endothelial growth factor regulates endothelial cell survival through the phosphatidylinositol 3′-kinase/Akt signal transduction pathway. Requirement for Flk-1/KDR activation. J Biol Chem 1998;273:30336-43.

29. Gerber H P, Dixit V, Ferrara N. Vascular endothelial growth factor induces expression of the antiapoptotic proteins Bcl-2 and A1 in vascular endothelial cells. J Biol Chem 1998;273:13313-6.

30. Alon T, Hemo I, Itin A, Pe'er J, Stone J, Keshet E. Vascular endothelial growth factor acts as a survival factor for newly formed retinal vessels and has implications for retinopathy of prematurity. Nat Med 1995; 10: 1024-8.

31. Tolle A, Kolleck I, Schiame M, Wauer R R, Stevens P A, Ruistow B: Effect of hyperoxia on the composition of the alveolar surfactant and the turnover of surfactant phospholipids, cholesterol, plasmalogens and vitamine E. Biochem Biophys Acta 1997; 1346:198-204.

32. Jehle R, Schlame M, Buttner C, Frey B, Sinha P, Rustow B. Platelet-activating factor (PAF)-acetylhydrolase and PAF-like compounds in the lung: effects of hyperoxia. Biochim Biophys Acta 2001; 1532:60-6.

33. Sabat R, Kolleck I, Witt W, Volk H, Sinha P, Rustow B. Immunological dysregulation of lung cells in response to vitamin E deficiency. Free Radic Biol Med 2001;30:1145-53.

34. Guthmann F, Kolleck I, Schachtrup C, Schlame M, Spener F, Rustow B. Vitamin E deficiency reduces surfactant lipid biosynthesis in alveolar type II cells. Free Radic Biol Med 2003; 1 5;34:663-73.

35. Hengartner M O: The biochemistry of apoptosis. Nature 2000;407:770-6.

36. Kolleck I, Wissel H, Guthman F, Schlame M, Sinha P, Rustow B. HDL-holoparticle uptake by alveolar type II cells: effect of vitamin E status. Am J Respir Cell Mol Biol 2002;27:57-63.

37. Rooney S A, Gobran L I. Alveolar lavage and lavaged lung tissue phosphatidylcholine composition during fetal rabbit development. Lipids 1977; 12:1050-4.

38. Guthmann F, Wissel H, Schachtrup C, Tolle A, Ruidiger M, Spener F, Rustow B. Hyperoxia of rats induces reversible pro-apoptosis in alveolar type II cells. Respir. Res. 2005;6:10.

39. Hideshima T, Nakamura N, Chauhan D, Anderson K C. Biologic sequelae of interleukin-6 induced PI3-K/Akt signaling in multiple myeloma. Oncogene 2001; 20:5991-6000.

40. Altavilla D, Saitta A, Guarini S, Galeano M, Squadrito G, Cucinotta G, Santamaria L B, Mazzeo A T, Campo G M, Ferlito M, et al.. Oxidative stress causes nuclear factor-κB activation in acute hypovolemic hemorrhagic shock. Free Rad. Biol. Med. 2001; 30:1055-1066.

41. Rüdiger M, Burkhardt W, Kraft S, Proquitte H, Kopke U, Wauer R R. Persurf—A new method for surfactant delivery. Biol Neonate 2003; 83:78

42. Gregor T, Schmalisch G, Burkhardt W, Proquitte H, Wauer R R, Ruidiger M. Aerosolization of perfluorocarbons during mechanical ventilation: an in vitro study. Intensive Care Med 2003; 29:1354-60.

43. Rüdiger M, Gregor T, Burkhardt W, Proquitte H, Wauer R R, Schmalisch G: Perfluorocarbon (PFC) species and nebulizer type influence aerosolization rate and particle size of PFC aerosol. J Crit Care 2004; 19:42-47

What is claimed is:

1. A method for treating a pulmonary disease or condition that is related to high concentrations of inspired oxygen, comprising administering to a patient in need thereof an effective amount of vascular endothelial growth factor (VEGF) or a pharmaceutically acceptable salt thereof.

2. The method of claim 1 wherein the VEGF is administered in a formulation comprising pharmaceutically acceptable carrier or excipient.

3. The method of claim 1 wherein the VEGF is a native human VEGF.

4. The method of claim 1 wherein the VEGF is a recombinant VEGF.

5. The method of claim 1 wherein the pulmonary disease or condition requires oxygen supplementation.

6. The method of claim 1 wherein the pulmonary disease or condition is caused by hyperoxia.

7. The method of claim 1 wherein the pulmonary disease or condition is caused by exposure to oxygen concentrations that are higher than under physiological conditions.

8. The method of claim 1 wherein the VEGF is administered by intra-tracheal administration.

9. The method of claim 1 wherein the VEGF is applied in a unit dose and a unit is in the range from 0.01 to 50 mg.

10. Method of claim 1 wherein the patient is on oxygen supplement and is being treated for prevention of acute and/or chronic damage to the lungs.

11. Method of claim 1 wherein the pulmonary disease being treated is selected from the group consisting of broncho-pulmonary dysplasia (BPD) of premature infants, respiratory distress syndrome, acute respiratory distress syndrome (ARDS) or neonatal respiratory distress syndrome (nRDS).

12. The method of claim 1, further comprising administering a surfactant.

13. The method of claim 1, further comprising administering a perfluorocarbon.

14. Method of claim 12 wherein said VEGF and said surfactant are administered concurrently or consecutively.

15. Method of claim 13 wherein said VEGF and said perfluorocarbon are administered concurrently or consecutively.

16. Method of claim 1 wherein the said patient is an adult, infant, neonate or preterm infant.


SEQUENCE LISTING (8 sequences)

SEQ ID NO: 1 - DNA, 20 bp, Rattus norvegicus
cagtgccagcctctgctcat

SEQ ID NO: 2 - DNA, 20 bp, Rattus norvegicus
atactcagcaccagcatcat

SEQ ID NO: 3 - DNA, 21 bp, Rattus norvegicus
tgcactggaccctggctttac

SEQ ID NO: 4 - DNA, 21 bp, Rattus norvegicus
cggcagtagcttcgctggtag

SEQ ID NO: 5 - DNA, 22 bp, Rattus norvegicus
tagctgtcgctctgtggttctg

SEQ ID NO: 6 - DNA, 22 bp, Rattus norvegicus
cctgcaagtaatctgaagggtt

SEQ ID NO: 7 - DNA, 21 bp, Rattus norvegicus
ggggccaccacgctcttctgt

SEQ ID NO: 8 - DNA, 21 bp, Rattus norvegicus
gcaaatcggctgacggtgtgg